(19) United States
(12) Patent Application Publication
(10) Pub. No.: US 2009/0042290 A1
(43) Pub. Date: Feb. 12, 2009

(54) METHOD OF MODIFYING A MACROMOLECULE WITHOUT PRIOR EXTRACTION FROM A SAMPLE

(76) Inventors: Christopher Steele, Manlius, NY (US); Darin Oppenheimer, Plainsboro, NJ (US); Sean Wuxiong Cao, Three Bridges, NJ (US); Carrie A. Trust, New Providence, NJ (US); George A. Green, IV, Newton, NJ (US); Jyoti Mehrotra, Bridgewater, NJ (US); Tatiana I. Vener, Stirling, NJ (US); Shobha A. Varde, Jacksonville, FL (US); Abhijit Mazumder, Basking Ridge, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/771,451

(22) Filed: Aug. 6, 2007

(51) Int. Cl. C12N 5/06; C07C 53/00; C07H 1/00; C07H 21/04; C07K 14/00

(52) U.S. Cl. 435/375; 435/270; 530/402; 536/1.11; 536/23.1; 536/23.7; 536/23.72; 554/1

(57) ABSTRACT

The present invention encompasses a method of modifying a macromolecule without prior extraction from a sample by converting the macromolecule in the sample with a chemical, removing or converting chemical intermediates, if necessary; and purifying the resulting modified macromolecule.

### STATEMENT REGARDING FEDERALLY SPONSORED RESEARCH OR DEVELOPMENT

No government funds were used to make this invention.

### BACKGROUND OF THE INVENTION

The present invention encompasses a method of modifying a macromolecule without prior extraction from a sample by converting the macromolecule in the sample with a chemical, removing or converting chemical intermediates, if necessary; and purifying the resulting modified macromolecule.

One method used by vertebrates and higher plants to regulation gene expression is the methylation of cytosines found in CpG islands located in promoter regions of various genes. In order to study this method of gene regulation, techniques were developed to discriminate methylated cytosines from unmethylated cytosines. One method is to chemically treat DNA in such a way that the cytosines are converted to uracils while 5-methyl-cytosines are not significantly converted. Frommer et al. (1992). A systematic investigation on the critical parameters of the modification procedure has also been made. Grunau et al. (2001). The treated DNA may be used as template for methylation specific PCR (MSP). DNA methylation and methods related thereto are discussed for instance in US patent publication numbers 20020197639, 20030022215, 20030032026, 20030082600, 20030087258, 20030096289, 20030129620, 20030148290, 20030157510, 20030170684, 20030215842, 20030224040, 20030232351, 20040023279, 20040038245, 20040048275, 20040072197, 20040086944, 20040101843, 20040115663, 20040132048, 20040137474, 20040146866, 20040146868, 20040152080, 20040171118, 20040203048, 20040241704, 20040248090, 20040248120, 20040265814, 20050009059, 20050019762, 20050026183, 20050053937, 20050064428, 20050069879, 20050079527, 20050089870, 20050130172, 20050153296, 20050196792, 20050208491, 20050208538, 20050214812, 20050233340, 20050239101, 20050260630, 20050266458, 20050287553 and U.S. Pat. Nos. 5,786,146, 6,214,556, 6,251,594, 6,331,393 and 6,335,165.

DNA modification kits are commercially available, they convert purified genomic DNA with unmethylated cytosines into genomic lacking unmethylated cytosines but with additional uracils. The treatment is a two-step chemical process consisting a deamination reaction facilitated by bisulfite and a desulfonation step facilitated by sodium hydroxide. Typically the deamination reaction is performed as a liquid and is terminated by incubation on ice followed by adding column binding buffer. Following solid phase binding and washing the DNA is eluted and the desulfonation reaction is performed in a liquid. Adding ethanol terminates the reaction and the modified DNA is cleaned up by precipitation. However, both commercially available kits (Zymo and Chemicon) perform the desulfonation reaction while the DNA is bound on the column and washing the column terminates the reaction. The treated DNA is eluted from the column ready for MSP assay. The modification is tedious and has many steps that cause yield loss and increase operator error. All of the available modification procedures begin with purified genomic DNA, which is a tedious process that also has many steps that cause yield loss and increase operator error.

### SUMMARY OF THE INVENTION

The present invention encompasses a method of modifying a macromolecule without prior extraction from a sample by converting the macromolecule in the sample with a chemical, removing or converting chemical intermediates, if necessary; and purifying the resulting modified macromolecule.

### BRIEF DESCRIPTION OF THE DRAWING

FIG. 1: Shows that DNA modification in without isolation from a biological sample is equivalent to such modification after isolation.

### DETAILED DESCRIPTION

The present invention encompasses a method of modifying a macromolecule without prior extraction from a sample by converting the macromolecule in the sample with a chemical, removing or converting chemical intermediates, if necessary; and purifying the resulting modified macromolecule.

The macromolecules can be any known in the art including, without limitation, DNA, RNA, cellular metabolites, lipids, carbohydrates and proteins. The DNA can be any known in the art including, without limitation, viral, nucleic, mitochondrial, plastid, bacterial and synthetic. The RNA can be any known in the art including, without limitation, rtRNA, tRNA, miRNA, rRNA and mRNA. The cellular metabolite can be any known in the art including, without limitation, those produced by a metabolic cycle or enzymatic effects. The lipid can be any known in the art including, without limitation, liposomes, cell membrane lipids, intracellular membrane lipids and extracellular lipids. The carbohydrate can be any known in the art including, without limitation, protein-bound carbohydrates and nucleic acid-bound carbohydrates. The protein can be any known in the art including, without limitation, intracellular and extracellular.

The modification is can be any known in the art including bisulfite and biotinylation of DNA or RNA, fluorination and methylation of RNA, heating, liposome formation, micelle formation, uni-layer formation and bilayer formation of lipids, oxidation, de-oxidation, amination and de-amination of carbohydrates and phosphorylation, dephosphorylation, methylation, biotinylation, amination, deamination, glycosylation and deglycosylation of proteins. 20070148670; Chuang et al. (2007); Emmerechts et al. (2007); Frommer et al. (1992); Grunau et al. (2001); Hurd et al. (2007); Jin et al. (2007); Oakeley (1999); Rathi et al. (2003); Rein et al. (1998); Sambrook et al. (2000); Wu et al. (2007).

The sample can be any known in the art including, without limitation, tissue, body fluid, a biopsy sample, and preserved tissue. The tissue can be any known in the art including, without limitation, whole organs, dissected organs, epithelium, neural, gastrointestinal, muscle, cardiac, mucosal and endothelium. The body fluid can be any known in the art including, without limitation, whole blood, plasma, urine, saliva, vitreous and serum. The biopsy sample can be any known in the art including, without limitation, fine needle aspirate, tissue section and skin sample. The preserved tissue can be any known in the art including, without limitation, fresh frozen, paraffin embedded and preserved in a preservation reagent. The preservation reagent can be any known in the art including, without limitation, formalin, RNAlater® and dimethylsulfoxide.

The purification can be any known in the art including, without limitation, particle-based, precipitation, centrifugation, electrophoretic and charge switch. The particle-based purification can be any known in the art including, without limitation, affinity, sizing and magnetic. The sizing particle can be any known in the art including, without limitation, silica-based and diatomaceous earth. The electrophoretic separation purification can be any known in the art including, without limitation, by size and/or charge. The electrophoretic separation purification can be any known in the art including, without limitation, by a gel formed of low molecular weight polymers and/or capillary.

The present invention provides a rapid and efficient method for obtaining bisulfite modified DNA. The method described herein effectively eliminates numerous steps of the previous methods thus reducing possible error while producing superior results. In addition considerable time savings of four to five hours are also realized.

The present invention provides a method of extracting and modifying DNA by obtaining a DNA sample; incubating the sample with an amount of a bisulfite and for a time and under conditions sufficient to convert at least ninety-five percent of the non-methylated cytosine residues in the DNA to uracil resides; binding the DNA in the sample to a column; washing the bound DNA to remove contaminants; incubating the column-bound DNA with a desulfonation reagent for a time and under conditions sufficient for desulfonation to occur; washing the bound DNA to remove the desulfonation reagent; and eluting the bisulfite modified DNA from the column.

The DNA can be at a concentration of from about 0.01 to about 30 μg and can be obtained by any method known in the art and can be purified DNA or DNA obtained directly from a cell lysate. For instance, the cell lysate can be formed from any suitable tissue by any method known in the art and directly treated with a bisulfite reagent. Cell lysis can be by for instance, proteinase and/or high salt concentration and/or detergent, sonication, freeze-thaw treatment or mechanical disruption. Any cell sample is suitable for use herein and can be obtained from tissue, body fluid, biopsy sample or preserved tissue.

The bisulfite reagent can be any known in the art, including, without limitation, sodium bisulfite or meta bisulfite. Other reagents are discussed for instance in US patent publications 20050089898, 20050095623 and 20050153308.

The incubation conditions of step b are about 1-16 hours at 50-95° C. with or without Thermocycling. Thermocycling can be for instance 3 hours at 70° C., 1 hour at 90° C. or cycling between 50° C. and 95° C.

The column can be any known in the art, preferably, it is silica-based or diatomaceous earth. The desulfonation can be by any method known in the art and is preferably performed with sodium hydroxide and an alcohol. Preferably, the alcohol is isopropanol or ethanol. More preferably, when the column is silica-based, the alcohol is ethanol and when the column is diatomaceous earth, the alcohol is isopropanol. The desulfonation preferably occurs from about 0-30, preferably about 5-15 and more preferably about 15 minutes at about 0° C. to about 50° C. Preferably, the temperature is about room temperature.

The modified macromolecule can be eluted by any method known in the art, including, without limitation with water or a suitable buffer.

The following examples are provided to illustrate but not limit the claimed invention. All references cited herein are hereby incorporated herein by reference.

### EXAMPLE 1

### Rapid Bisulfite Modification of DNA from a Cell Lysate Obtained from Fresh Frozen Paraffin Embedded Tissue

Microfuge tube containing cell culture pellet in PBS
Add 100 μl DNA extraction buffer (75 mM NaCl, 25 mM EDTA, and 0.5% Tween 20), incubate 56° C. for 10 minutes.
Proceed with 3M NaOH addition described below
   Or a microfuge tube containing (up to) 5 10 micron FFPE block slices.
   The FFPE slices require deparaffination:
Add 1 ml of Xylene to the tube and mix by inverting several times
Incubate at RT for 5 min and centrifuge at full speed for 5 minutes at RT
Remove the supernatant without removing any pellet
Repeat the 1 ml Xylene extraction
Add 1 ml EtOH (100%) to the pellet and gently mix by inverting
Centrifuge full speed for 5 minutes at RT
Carefully remove the EtOH by pipetting without removing any of the pellet
Repeat the EtOH wash
Dry the pellet in a 37° C. heat block with the microfuge tubes open
   Extraction/modification procedure for FFPE samples
Add 90 μl extraction buffer (10 mM Tris, pH 8.0, 150 mM NaCl, 2 mM EDTA, and 0.5% SDS) and 10 μl proteinase K and incubate overnight at 56° C.
After incubation the extract should be clear, if not add 10 μproteinase K and incubate for 1 hr
Repeat until lysis is clear and proceed with 3M NaOH addition described below.
Add 1/10 volume 3M NaOH and incubate 56° C. for 10 minutes.
Add 2 volumes of a saturated solution of sodium bisulfite pH 5.0 with 10 mM hydroquinone, incubate at 70° C. for 3 hours in the dark.
Stop the reaction by incubating on ice for 10 minutes. Add 0.5 volumes of Isopropanol and gently vortex or pipette up and down.
Add the sample to a Qiagen DNA purification column (QiaAmp DNA purification kit), spin 1 min and empty waste tube;
Add 500 μl AW1 wash buffer, spin 1 min and empty waste tube;
Add 500 μl AW2 wash buffer, spin 1 min and empty waste tube;
Add 200 μl desulfonation buffer (300 mM NaOH, 80% Isopropanol), incubate 10 min room temperature, spin 1 min and empty waste tube;
Add 500 μl AW2 wash buffer, spin 1 min, empty waste tube and spin 1 min,
Add 500 μl AW2 wash buffer, spin 1 min, empty waste tube and spin 1 min,
Elute with 50 μl TE, spin 1 min into new 1.5 ml microfuge tube and store at −20° C.

The efficiency of the procedure is assayed using quantitative PCR and β-Actin using GSTP1 as markers. The β-Actin promoter is not methylated and the marker is designed to serve as a control for the modification procedure. The Ct value produced by this marker is reflective of the number of genome equivalents added to the assay. Whereas the GSTP1 promoter is methylated epigenetically and may be reflective of a cancerous state. Therefore the GHSTP1 marker Ct value is more variable and usually greater than the β-Actin Ct value. The Prostate FFPE blocks were obtained from Asterand. The “Zymo” treatment refers commercially available DNA modification kit sold as EZ DNA Methylation Kit from Zymo Research. A 2 Step procedure refers to two separate procedures that use a DNA purification kit (Qiagen QiaAmp mini DBNA purification kit) and a DNA modification kit such as the Zymo kit. The Zymo 1 step procedure is identical to the ID procedure until the 3M NaOH step where the Zymo DNA modification procedure is followed replacing the 3M NaOH with M-Dilution buffer.

The results shown in Table 1 using cell culture pellets indicate that the 1 Step method produces lower Ct values and thus superior results when compared to the Qiagen/Zymo 2 Step procedure. Statistically, a paired T test indicates the methods, using the β-Actin marker, are significantly different from each other with a P value of 0.00006 while the GSTP1 marker had a P value of 0.0001. The results shown in Table 2 indicate that ID 1 step produces lower Ct values when compared to the Zymo 1 Step method with the β-Actin marker produces a P value of 0.02 and the GSTP1 marker results in a P value of 0.003

However, the results shown in Table 1 using prostate FFPE blocks indicate the opposite results from the cell cultures with the Zymo 2 Step producing lower Ct values then the 1 step method. The β-Actin marker results indicate that the methods are significantly different with a P value of 0.021 while the GSTP1 results suggest that they maybe different with a P value of 0.054. The different results suggested that the extraction buffer for cell culture might not be sufficient to lysis tissue blocks. Table 4 results compares the ID 1 Step and the Zymo 1 Step methods using a more aggressive extraction buffer switching the Tween to SDS while using prostate tissue blocks. The ID 1 Step method once again shows superior results suggesting that the failure to produce superior results in Table 3 was due to the extraction buffer. The β-Actin marker results indicates that the methods are significantly different with a P value of 0.0008 while the GSTP1 results suggest that they maybe different with a P value of 0.056. Since the QPCR assay method only has 40 cycles, once an assay fails to produce Ct's then it is not significant to compare with assays that produce Ct's. If the two samples that had assays that failed then the P values would be 0.0025, which indicates the methods using the GSTP1 marker are significantly different.

TABLE 1
Results comparing the 1 Step procedure to the Qiagen/Zymo (Zymo) 2
Step procedure
Cell Culture LnCAP 1 × 10⁵ Cell pellets, 2 pellets tested in duplicate

| Treatment | Procedure | Marker | Ct | SD | P value |
|---|---|---|---|---|---|
| ID | 1 Step | β-Actin | 26.94 | 0.24 | 0.00006 |
| Zymo | 2 Step | β-Actin | 27.80 | 0.29 | |
| ID | 1 Step | GSTP1 | 27.66 | 0.20 | 0.0001 |
| Zymo | 2 Step | GSTP1 | 28.54 | 0.23 | |

TABLE 2
Results comparing the 1 Step procedure to the Zymo 1 Step procedure
Cell Culture LnCAP 1 × 10⁵ Cell pellets, 2 pellets tested in duplicate

| Treatment | Procedure | Marker | Ct | SD | P value |
|---|---|---|---|---|---|
| ID | 1 Step | β-Actin | 27.60 | 0.18 | 0.020 |
| Zymo | 1 Step | β-Actin | 29.90 | 0.08 | |
| ID | 1 Step | GSTP1 | 28.48 | 0.07 | 0.003 |
| Zymo | 1 Step | GSTP1 | 30.87 | 0.18 | |

TABLE 3
Results comparing the 1 Step procedure to the Qiagen/Zymo (Zymo) 2
Step procedure
Prostate FFPE Blocks Using The Cell Culture Extraction Buffer

| | Tissue | | Average | | Average | |
|---|---|---|---|---|---|---|
| Treatment | Block | Procedure | β-Actin Ct | SD | GSTP1 Ct | SD |
| ID | 1 | 1 Step | 30.68 | 0.08 | 32.64 | 0.27 |
| ID | 2 | 1 Step | 33.84 | 0.22 | 37.20 | 0.33 |
| ID | 3 | 1 Step | 31.26 | 0.04 | 35.19 | 0.16 |
| ID | 4 | 1 Step | 34.21 | 0.03 | 38.57 | 0.08 |
| Zymo | 1 | 2 Step | 29.68 | 0.19 | 33.40 | 0.07 |
| Zymo | 2 | 2 Step | 28.72 | 0.04 | 32.87 | 0.19 |
| Zymo | 3 | 2 Step | 27.69 | 0.01 | 32.11 | 0.22 |
| Zymo | 4 | 2 Step | 27.62 | 0.04 | 33.65 | 0.25 |

| | | |
|---|---|---|
| P value | 0.021 | 00.054 |

TABLE 4
Results comparing the 1 Step procedure to the Zymo 1 Step procedure
using Prostate FFPE blocks.
Prostate FFPE Blocks Usin The FFPE Extraction Buffer

| | | β-Actin | β-Actin | GSTP1 | GSTP1 |
|---|---|---|---|---|---|
| Treatment | Tissue Block | Ct | SD | Ct | SD |
| Zymo | 12 | 35.16 | 0.10 | 40 | 0.00 |
| ID | 12 | 31.21 | 0.04 | 40 | 0.00 |
| Zymo | 16 | 34.41 | 0.12 | 35.76 | 0.79 |
| ID | 16 | 30.23 | 0.33 | 31.98 | 0.25 |
| Zymo | 18 | 35.69 | 0.13 | 40.00 | 0.00 |
| ID | 18 | 32.63 | 0.16 | 39.27 | 1.03 |
| Zymo | 20 | 33.08 | 0.17 | 36.07 | 0.05 |
| ID | 20 | 28.29 | 0.03 | 32.34 | 0.19 |
| Control | Plasmid | 23.15 | 0.79 | 22.86 | 0.34 |

| | | |
|---|---|---|
| P value | 00.0008 | 00.056 |

### EXAMPLE 2

### Bisulfite Treatment of DNA Obtained from Urine

The purpose of this experiment was to compare the DEM kit versus the Zymo EZ Modification kit on purified DNA obtained from LnCAP cells and urine. Ten normalized random samples were divided between the two kits

5 Samples per Kit (DEM and Zymo)

PCR performed in duplicate using Fast Start Taq (GSTP1, β-Actin, and APC)

ANOVA analysis indicates a P value of 0.663 and a Paired T-Test indicates that there is no statistical difference between DEM and Zymo for GSTP1.

ANOVA analysis indicates a P value of 0.008 and a Paired T-Test indicates that there is a statistical difference between DEM and Zymo for β-Actin.

The results indicate that Zymo and the DEM kit are equivalent for GSTP1

The DEM kit was optimized for tissue as opposed to purified DNA further optimization within ProMU could lead to lower CT values.

The DEM kit demonstrates a higher β-Actin value

| β Actin | GSTP1 |
|---|---|
| One Way ANOVA P = 0.000 | One Way ANOVA P = 0.663 |
| DEM (Mean CT) 34.990 | DEM (Mean CT) 31.30 |
| DEM STDEV 1.341 | DEM STDEV 0.839 |
| Zymo (Mean CT) 33.270 | Zymo (Mean CT) 31.140 |
| Zymo STDEV 1.240 | Zymo STDEV 0.773 |

| Number | Kit | β-Actin | GSTP1 | APC |
|---|---|---|---|---|
| 1 | DEM | 33.8 | 30.4 | 34.0 |
| 2 | DEM | 34.8 | 30.5 | 33.2 |
| 3 | DEM | 37.8 | 31.8 | 34.1 |
| 4 | DEM | 35.1 | 32.2 | 33.9 |
| 5 | DEM | 35.7 | 32.5 | 34.4 |
| 6 | DEM | 35.6 | 32.2 | 34.3 |
| 7 | DEM | 33.4 | 30.5 | 32.4 |
| 8 | DEM | 33.4 | 30.4 | 32.3 |
| 9 | DEM | 35.8 | 31.5 | 33.3 |
| 10 | DEM | 34.5 | 31.0 | 32.4 |
| 1 | Zymo | 32.7 | 32.4 | 33.4 |
| 2 | Zymo | 32.3 | 30.8 | 31.5 |
| 3 | Zymo | 35.6 | 31.9 | 33.3 |
| 4 | Zymo | 34.5 | 31.8 | 33.2 |
| 5 | Zymo | 34.2 | 31.6 | 32.6 |
| 6 | Zymo | 34.2 | 31.4 | 32.5 |
| 7 | Zymo | 32.1 | 30.4 | 31.5 |
| 8 | Zymo | 32.2 | 30.5 | 31.5 |
| 9 | Zymo | 32.3 | 30.2 | 31.6 |
| 10 | Zymo | 32.6 | 30.4 | 31.6 |

The binding of crude lysate containing DNA or purified genomic DNA is uniquely bound to the silica-gel based column utilizing the high concentration of salt that is present from the bisulfite conversion.

Ethanol is added to the sample prior to binding only to dissolve the conversion reagent.
Add 1/10 volume M-Dilution Buffer and incubate 70° C. for 20 minutes to chemically denature the double stranded DNA
Add 2 volumes of CT conversion reagent (Zymo), incubate at 70° C. for 3 hours in the dark.
Add 0.5 volumes of ethanol (100%) and gently vortex or pipette up and down.
   Microfuge tube containing purified DNA from urine at a starting volume of 45 μl
Proceed with M-Dilution Buffer addition described below
Or a microfuge tube containing (up to) 5 10 micron FFPE block slices of a formalin fixed paraffin embedded cell culture pellet which is known to express GSTP1 hyper Methylation.
   The FFPE slices require deparaffination:
Add 1 ml of Xylene to the tube and mix by inverting several times
Incubate at RT for 5 min and centrifuge at 13,200 rpm for 5 min at RT
Remove the supernatant without removing any pellet
Repeat the 1 ml Xylene extraction
Add 1 ml EtOH (100%) to the pellet and gently mix by inverting
Centrifuge at 13,200 rpm for 5 min at RT
Carefully remove the EtOH by pipetting without removing any of the pellet
Repeat the EtOH wash
Dry the pellet and remove the residual ethanol in a 37° C. heat block with the microfuge tubes open for approximately 10 minutes
   Extraction/modification procedure for FFPE samples
Add 35 μl buffer ATL (Qiagen) and 10 μl of proteinase K (Qiagen) and incubate overnight at 56° C.
After incubation the extract should be clear and homogeneous
Proceed with M-Dilution Buffer (Zymo Research) addition described below
Add the sample to a Qiagen DNA purification column (QiaAmp Micro DNA purification kit), spin 1 min at 13,200 rpm and empty waste tube;
Add 500 μl AW1 wash buffer, spin 1 min at 13,200 rpm and empty waste tube;
Add 200 μl desulfonation buffer (300 mM NaOH, 90% Ethanol), incubate 20 min room temperature, spin 1 min at 13,200 rpm and empty waste tube;
Add 500 μl AW2 wash buffer, spin 1 min (13,200 rpm), empty waste tube and spin 3 min (13,200 rpm).
Elute with 20-25 μl buffer AE, TE, or Nuclease Free Water. Incubate column for three min at room temperature and spin 1 min into new 1.5 ml microfuge tube and store at −20° C. or −80° C. foe

### EXAMPLE 3

### Large Scale DNA Modification

Large scale DNA modification may be necessary to make panels for quality control testing of methylation specific PCR methods and kits.

1. Denature 20 μg of Prostate Cell Culture Cell line 22Rv1 genomic DNA (ATCC) in a total volume of 225 μl TE, plus 27.5 μl of a 3.0 M NaOH solution. Incubate 10 minutes 37° C.

2. Add 2× volume Conversion reagent (Zymo); incubate 3 hr 70° C. followed by 10 min on ice.

Bind the DNA to a solid phase support by adding 2 ml of binding buffer containing the support matrix (Promega) and adding it to the syringe column vacuum apparatus. Using the vacuum, filter the matrix.

Using the vacuum, wash with the matrix with 1 ml 80% IPA

Add 2 ml OD desulfonation buffer (0.3 M NaOH in 80% IPA) and incubate at room temperature for 10 minutes. Using the vacuum, remove the buffer.

Using the vacuum, wash with 1 ml 80% IPA.

3. Remove the column from the syringe and place it in a 1.5 ml microfuge tube Elute 5×200 μl followed by addition of 1 ml EB Table 5 depicts the results obtained.

TABLE 5

| Marker | Average Ct | SD |
|---|---|---|
| Actin | 33.1 | 0.2 |
| GSTP1 | 0 | |
| APC | 31.1 | 0.2 |

In this cell line the GSTP1 promoter is known to be unmethylated therefore the lack of Ct values for this marker is expected.

### EXAMPLE 4

A Modified Protocol for Fast and Efficient Bisulfite Modification of Genomic DNA

The EZ DNA Methylation Kit is provided by Zymo Research (Orange, Calif.) to perform bisulfite modification of DNA. As per manufacturer's recommendation the DNA sample to be modified is incubated with the bisulfite conversion reagent at 50° C. for 12-16 hrs. These conditions have been modified to generate comparable quality bisulfite converted DNA in much less time. Several temperatures for different times were tested and demonstrated that incubation of DNA sample with bisulfite conversion reagent at 70° C. for 1-3 hr provides efficient bisulfite modification comparable to modification conditions recommended in the kit. The data below show methylation specific PCR analysis with DNA samples incubated with bisulfite reagent at different temperatures for different times.

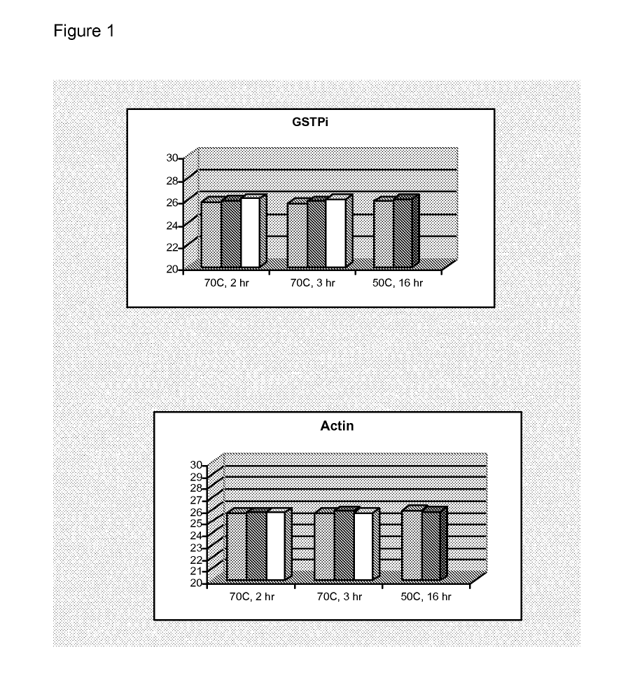

FIG. 1 shows that Veridex modified protocol of conversion at 70° C., 2 or 3 hr is equivalent to manufacturer recommended 50° C. for 16 hrs. This innovation makes this procedure much faster.

### EXAMPLE 5

### A Quick and Efficient Protocol for DNA Extraction and Modification from Paraffin Embedded Tissue

Extraction of genomic DNA and its bisulfite modification prior to being used in a MSP reaction comprise very significant upstream procedures that are part of this in vitro diagnostic assay. These procedures can be time consuming involving many tedious steps and could also increase chances of sample contamination. By combining the use of a lysis buffer, 10 mM Tris pH 8.0, 150 mM NaCl, 2 mM EDTA, 0.5% SDS including proteinase K and a bisulfite modification kit, a quick and simple sample processing protocol to recover and modify minimal amounts of DNA available from these sample types has been developed.

The following steps were performed:
1. FFPE tissue (Biopsies) (5×10μ sections) are placed in Eppendorf tubes.
2. Spin the tube briefly and Add 500 μl Xylene, vortex briefly at medium speed.
3. Incubate at RT for 10 min. (During incubation at least at 2 intervals mix the sample by inverting several times).
4. Centrifuge at full speed for 10 min at room temperature.
5. Remove supernatant by carefully decanting the liquid without losing the pellet.
6. Add 500 μl ethanol (100%, 200 proof) to the pellet to remove residual Xylene. Vortex briefly at medium speed and let the tubes stand for 5 min, mix by inverting several times.
7. Centrifuge at full speed for 10 min at room temperature.
8. Remove the ethanol by carefully decanting the liquid without losing the pellet.
9. Repeat steps 7-10. Make sure to decant most of the liquid in this step.
10. Incubate the open microcentrifuge tube at 50-55° C. for 10-15 min in an oven to evaporate residual ethanol. Before moving to next step make sure all ethanol has evaporated. If not incubate longer.
11. Add 40 μl of TNES lysis buffer (10 mM Tris pH 8.0, 150 mM NaCl, 2 mM EDTA, 0.5% SDS) and suspend the tissue by flicking the tube.
12. Add 10 μl Proteinase K (20 mg/ml), vortex briefly, spin very briefly.
13. Incubate the sample at 56° C. O/N in a heat block with shaking at 500 rpm.
14. Next morning spin the tubes briefly and check the tissue for complete digestion. If there is any left over tissue add 2 μl fresh Proteinase K (20 mg/ml), mix by gentle vortexing and incubate another 1 hr at 56° C., 500 rpm in heat block.
15. Incubate tubes at 70° C.×10 min on the heat block, spin and store at −20° C. for long-term storage or proceed directly for Bisulfite modification of extracted DNA using a commercially available DNA modification kit from ZymoResearch.
16. Add 5 μl of M-Dilution Buffer directly to 45 μl of tissue lysate
17. Mix sample by flicking or pipetting up and down. Spin the sample briefly. Incubate the sample at 37° C. for 15 minutes in a heat block with shaking at 1100 rpm.

During the 15 min incubation, prepare CT Conversion Reagent (as per manufacturer's instructions).
18. After the above 15 minutes incubation, add 100 μl of the prepared CT Conversion Reagent (after briefly spinning) to each sample and vortex lightly (The sample may turn cloudy). Spin the sample briefly. Incubate the sample at 70° C. for 3 hr with the heating block (shaking at 1100 rpm) covered with aluminum foil. (The CT Conversion Reagent is light sensitive, so try to minimize reaction's exposure to light).
19. Spin the sample down briefly. Incubate the sample on ice for 10 min.
20. Add 400 μl of M-Binding buffer to the sample and mix by pipetting up and down
21. Load all the supernatant (including any precipitate) onto a Zymo-Spin I Column and place column into a 2 ml collection tube and centrifuge at maximum speed for 15-30 seconds. Discard the flow-through
22. Add 200 μl of M-Wash Buffer to the column.
23. Centrifuge at maximum speed for 15-30 seconds. Discard the flow-through.
24. Add 200 μl of M-Desulfonation Buffer to the column and let the column stand at room temperature for 15 minutes.
25. Centrifuge at maximum speed for 15-30 seconds. Discard the flow-through.
26. Add 200 μl of M-Wash Buffer to the column.
27. Centrifuge at maximum speed for 15-30 seconds.
28. Add another 200 μl of M-Wash Buffer to the column.
29. Centrifuge at maximum speed for 30 sec (A longer spinning duration for this last wash is necessary for complete removal of wash buffer residues). Discard the flow-through.
30. Place the column into a clean 1.5 ml tube.
31. Add 25 μl of M-elution buffer directly to the column matrix. Let the column stand for 1 min at RT. Centrifuge at maximum speed for 1 minute to elute the DNA.
32. Store the eluted DNA at −80° C.
33. Use 5 μl in MSP reaction.

The protocol described above excludes the use of a DNA purification kit prior to bisulfite modification, thereby, reducing sample processing times, preventing DNA losses during purification, reducing cost, and reducing chances of contamination.

Table 6 shows that using the tissue lysate directly for bisulfite modification of DNA gives comparable and even lower Cts than purified DNA using Qiagen DNA isolation kit. Thus further purification of DNA is not required prior to DNA modification using ZymoResearch EZ DNA methylation kit. Also, the data show that combining TNES/PK digestion and EZ DNA methylation kit yields more DNA sample.

TABLE 6

| FFPE Tissue | DNA extraction procedure | β-actin Ct | GSTP1 Ct |
|---|---|---|---|
| 2 × 5μ sections | Qiagen DNA isolation kit | 30.1 | 30.9 |
| 2 × 5μ sections | TNES/PK digestion | 25.4 | 26.7 |

Table 7 shows that direct lysate from FFPE biopsy tissue can be used successfully for downstream DNA modification with comparable and even better results as compared to using Qiagen DNA isolation kit, thus avoiding unnecessary DNA purification steps and losses.

TABLE 7

| | β actin Av Cts | β actin Av Cts |
|---|---|---|
| Prostate core Biopsies | TNES/PK protocol | Qiagen DNA isolation |
| Sample prep | (40 micron) | (50 micron) |
| Zymo elution vol. | 25 μl | 50 μl |
| Input in PCR | 5 μl | 5 μl |
| 60A | 34.45 | 33.95 |
| 60B1 | 32.75 | 34.30 |
| 60B2 | 32.90 | 33.25 |
| 64A | 33.30 | 34.20 |
| 64B | 35.70 | undetermined |

DNA methylation assay is developed to be used on patient samples such as archived formalin-fixed, paraffin-embedded tissues, freshly collected urine and blood samples that comprise an invaluable resource for translational studies of cancer and a variety of other diseases. Sample processing is a key upstream part of this diagnostic assay. Conventionally, DNA is purified from these sample types by using standard phenol-chloroform extraction or column based procedures and then subjected to bisulfite modification procedures. Several commercial kits exist for purification of DNA from paraffin embedded archived tissues, and body fluids. However, loses during such extensive purification procedures can significantly reduce DNA yields when very small amount of starting tissue or body fluid sample type is available. Low DNA yields can severely impact the downstream assay performance and can also be time consuming. To avoid DNA loses some studies have used digested tissue lysate directly for bisulfite modification of genomic DNA using standard in solution bisulfite modification protocols. The protocol developed in this invention (referred to as TNES protocol) quickly and efficiently extracts and bisulfite modifies genomic DNA by using the deproteinized lysed tissue extract or lysed cells from urine sediment and directly using this with a commercially available DNA methylation kit such as ZymoResearch EZ for downstream bisulfite modification without any further purification steps. In addition, the present invention improves multiplex PCR assay performance by minimizing loss during additional purification steps.

### EXAMPLE 6

### Processing DNA Samples from Urine

A. Protocol for DNA Extraction from Urine Samples (TNES Protocol)
1. After collection, urine is to be maintained at 4° C. until processed.
2. Urine is placed into a labeled 50 ml Falcon polypropylene tube and LNCap cells (10,000 cells/50 ml) representing shedded tumor cells in a prostate cancer patient are spiked per tube. Then the cells and particulates are pelleted by centrifugation at 3,000×g at 4° C.
3. Following centrifugation, carefully decant the urine supernatant into sterile labeled 50 ml tube and perform pellet washings in 2 steps.
4. For the first wash, the pellet is washed at 4° C. with 40 ml of cold PBS in the original 50 ml tube, gently invert the tube several times to resuspend the pellet, and centrifuge at 3,000×g. Aspirate the PBS wash using a vacuum attached to a long narrow glass or plastic tube (drawn-out Pasteur pipette or long plastic tip) to remove as much of the wash as possible and to prevent dislodging of the pellet over a large area of the tube (discard wash).
5. For the second wash, the pellet is resuspended in a smaller volume of 1 ml of cold PBS by gently pipetting up and down with a Pipetman. Once the pellet is suspended, then transfer from the 50 ml tube to a 1.5 ml microcentrifuge tube. With an additional 0.4 ml PBS, rinse the tip and the 50 ml tube to recover as much of pellet as possible and combine with the original 1 ml in the 1.5 ml tube.
6. Centrifuge at 10,000×g for 5 min and remove the wash by vacuum aspiration with a drawn out pipette/plastic tip. Use slight tilting of the tube to remove as much of the liquid as possible (discard wash). The tube containing the washed urine pellet is then placed into the same box as the 50 ml tube of clarified urine and stored at −20° C. until shipping.
7. To ˜100 μl of washed pellet add 100 μl TNES lysis buffer (10 mM Tris pH 8.0, 150 mM NaCl, 2 mM EDTA, 0.5% SDS), incubated @ 56° C. for 30 min. and then stored at −20° C. until processing with bisulfite modification kit (see below, part 11). In case of cells only, 20 μl TNES buffer was added to 20 μl cell suspension.
   II. Sodium Bisulfite Modification of genomic DNA using EZ-DNA methylation kit from ZymoResearch

The EZ DNA Methylation Kit is provided by Zymo Research (Orange, Calif.) to perform bisulfite modification of DNA. As per manufacturer's recommendation the DNA sample to be modified is incubated with the bisulfite conversion reagent at 50° C. for 12-16 hrs. These conditions have now been modified to generate comparable quality bisulfite converted DNA in much less time. Several temperatures and different times were tested and it was demonstrated that incubation of DNA sample with bisulfite conversion reagent at 70° C. for 1-3 hr provides efficient bisulfite modification comparable to modification conditions recommended in the kit

The protocol is as follows for processing urine samples:

M-Wash Buffer (Prepare before starting using the kit)
Preparation of M-Wash buffer: Add 24 ml absolute ethanol to the M-Wash buffer Concentrate to make the final M-Wash buffer for D5001 (Use 96 ml Ethanol for D5002).

### 1. DNA Modification Procedure

a. Add 5 μl of M-Dilution Buffer directly to 45 μl of urine lysate or for the higher sample volume scale up M-Dilution Buffer and urine crude lysate proportionally. For instance, for 150 μl urine lysate add 20 μM-dilution buffer and 30 μl water (total of 200 μl).
b. Mix sample by flicking or pipetting up and down. Spin the sample briefly. Incubate the sample at 37° C. for 15 min in a heat block with shaking at 1100 rpm.
   During the 15 min incubation, prepare CT Conversion Reagent (as per manufacturer's instructions).
c. After the above 15 minutes incubation, add 100 μl of the prepared CT

Conversion Reagent (after briefly spinning) to each sample (or add 400 μl CT reagent for a scaled up protocol) and vortex lightly (the sample may turn cloudy). Spin the sample briefly. Incubate the sample at 70° C. for 3 hr with the heating block (shaking at 1100 rpm) covered with aluminum foil. (The CT Conversion Reagent is light sensitive, so try to minimize reaction's exposure to light).

### 2. Desalting

a. Spin the sample down briefly. Incubate the sample on ice for 10 min. In the case of the higher volume of urine sample, aliquot into two tubes of 300 μl each.
b. Add 400 μl of M-Binding buffer to the sample and mix by pipetting up and down or for scaled up urine samples add 800 μl of M-Binding buffer to each aliquot, mixed and quickly spun.
c. Load all the supernatant (including any precipitate) onto a Zymo-Spin I

Column and place column into a 2 ml collection tube and centrifuge at maximum speed for 15-30 seconds. Discard the flow-through. For a higher sample volume, this step is repeated by adding supernatant from each aliquoted tube one at a time on the column and centrifuging until all of the sample is loaded onto the column.
d. Add 200 μl of M-Wash Buffer to the column.
e. Centrifuge at maximum speed for 15-30 seconds. Discard the flow-through.
   3. Desulfonation, 2^{nd} desalting and elution
a. Add 200 μl of M-Desulfonation Buffer to the column and let the column stand at room temperature for 15 minutes.
b. Centrifuge at maximum speed for 15-30 seconds. Discard the flow-through.
c. Add 200 μl of M-Wash Buffer to the column.
d. Centrifuge at maximum speed for 15-30 seconds.
e. Add another 200 μl of M-Wash Buffer to the column.
f. Centrifuge at maximum speed for 30 sec (a longer spinning duration for this last wash is necessary for complete removal of wash buffer residues). Discard the flow-through.
g. Place the column into a clean 1.5 ml tube.
h. Add 25 μl of M-elution buffer directly to the column matrix. Let the column stand for 1 min at RT. Centrifuge at maximum speed for 1 minute to elute the DNA.
i. Store the eluted DNA at −80° C.
j. Use 5 μl in MSP reaction.

### Results:

The protocol described above excludes the use of a DNA purification kit prior to bisulfite modification, thereby, reducing sample processing times, preventing DNA losses during purification, reducing cost, and reducing chances of contamination).

Table 8 shows end results from MSP assay on Cepheid Smart Cycler with DNA samples processed by TNES protocol from 50 ml of urine. Better β-actin and GSTP1 Cts (up to 3 Cts lower) are observed using above described TNES/PK digestion protocol over use of purified DNA using commercially available Qiagen DNA isolation kit (QiAmp Viral RNA kit). Thus further purification of DNA is not required prior to DNA modification using this method. Also, the data show that combining TNES/PK digestion and EZ DNA methylation kit yields more DNA sample.

TABLE 8
TNES protocol vs. Qiagen DNA isolation kit
(10,000 LNCaP cells/50 ml urine)

| | extraction | Av | |
|---|---|---|---|
| INPUT | protocol | β-actin Ct | Av GSTP1 Ct |
| 50 ml Urine/10⁴ LNCaP cells | TNES | 29.9 | 32.9 |
| 50 ml Urine/10⁴ LNCaP cells | Qiagen | 30.9 | 34.6 |
| 10⁴ LNCaP cells | TNES | 32.15 | 30.6 |
| 10⁴ LNCaP cells | Qiagen | 35.3 | 33.7 |

Results with TNES extraction protocol are even more compelling on serial dilutions of LNCaP prostate cells (range of 10,000 to 100 spiked per 50 ml pooled urine from healthy donors). Combined protocol for DNA extraction followed directly by bisulfite modification allows to improve sensitivity of the prostate methylation assay by 10 fold (Table 9) as compared with two commercial kits combined. TNES protocol allows detection of 100 cells per 50 ml urine, the level which is undetectable by Qiagen protocol using both Ct value and copy number analysis.

TABLE 9
TNES protocol vs. Qiagen DNA isolation kit (LNCaP cells: 10,000,
1000 and 100/50 ml urine).

| TNES-ZR BMK protocol | | | | Qiagen-ZR BMK protocol | | |
|---|---|---|---|---|---|---|
| Task | | | Ratio Gst-Pi | | | Ratio Gst-Pi |
| # cells/ml | | GSTP | M/β-actin | | GSTP1 | M/β-actin |
| urine | GSTP Ct | Copies | (copies) × 1000 | GSTP1 Ct | Copies | (copies) × 1000 |
| 10⁴/50 | 29.70 | 6365 | 487 | 30.88 | 3051 | 575 |
| 10⁴/50 | 31.10 | 2651 | 113 | 33.63 | 546 | 68 |
| 10⁴/50 | 31.38 | 2232 | 126 | 33.83 | 482 | 58 |
| 10³/50 | 35.05 | 224 | 17 | 38.93 | 20 | 2 |
| 10³/50 | 34.43 | 331 | 22 | 38.58 | 25 | 3 |
| 10²/50 | 38.60 | 24 | 2 | 00.00 | 0 | 0 |
| 10²/50 | 0.00 | 0 | 0 | 00.00 | 0 | 0 |

Ct values, Copy numbers and methylation ratios are presented for sample replicates of the same cell load in 50 ml urine

Although the foregoing invention has been described in some detail by way of illustration and example for purposes of clarity of understanding, the descriptions and examples should not be construed as limiting the scope of the invention.

### REFERENCES

20020197639, 20030022215, 20030032026, 20030082600, 20030087258, 20030096289, 20030129620, 20030148290, 20030157510, 20030170684, 20030215842, 20030224040, 20030232351, 20040023279, 20040038245, 20040048275, 20040072197, 20040086944, 20040101843, 20040115663, 20040132048, 20040137474, 20040146866, 20040146868, 20040152080, 20040171118, 20040203048, 20040241704, 20040248090, 20040248120, 20040265814, 20050009059, 20050019762, 20050026183, 20050053937, 20050064428, 20050069879, 20050079527, 20050089870, 20050130172, 20050153296, 20050196792, 20050208491, 20050208538, 20050214812, 20050233340, 20050239101, 20050260630, 20050266458, 20050287553, 20070148670 and U.S. Pat. Nos. 5,786,146, 6,214,556, 6,251,594, 6,331,393 and 6,335,165
Chuang et al. (2007) Epigenetics and microRNAs Ped Res 61:24R-29R
Emmerechts et al. (2007) Post-transcriptional modification mapping in the Colstridium acetobutylicum 16S rRNA by mass spectrometry and reverse transcriptase assays Nucl Acids Res 35:3494-3503
Frommer et al. (1992) A genomic sequencing protocol that yields a positive display of 5-methylcytosine residues in individual DNA strands Proc Natl Acad Sci USA 89:1827-31
Grunau et al. (2001) Bisulfite genomic sequencing: systematic investigation of critical experimental parameters Nucl Acids Res 29:E65-5.
Hurd et al. (2007) Detection of ROS-sensitive thiol proteins by redox-difference gel electrophoresis (Redox-DIGE) J Biol Chem (in press) epub
Jin et al. (2007) Influence or stereochemistry and redox potentials on the singles- and double-strand DNA cleavage efficiency of Cu(II) and Ni(II) Lys-Gly-His-derived ATCUN metallopeptides JACS 129:8353-8361
Oakeley (1999) DNA methylation analysis: a review of current methodologies Pharmacol Thera 84:389-400
Rathi et al. (2003) Aberrant methylation of the HIC1 promoter is a frequent event in specific pediatric neoplasms Clin Cancer Res 9:3674-33678
Rein et al. (1998) Identifying 5-methylcytosine and related modifications in DNA genomes Nucl Acids Res 26:2255-2264
Sambrook et al. (2000) Molecular Cloning: A Laboratory Manual (3^{rd} Ed) Cold Spring Harbor Laboratories
Wu et al. (2007) Computer modeling of mitochondrial TCA cycle, oxidative phosphorylation, metabolite transport, and electrophysiology J Biol Chem (in press) epub

1. A method of modifying a macromolecule without prior extraction from a sample comprising the steps of
a. converting the macromolecule in the sample with a chemical
b. removing or converting chemical intermediates, if necessary; and
c. purifying the resulting modified macromolecule.

2. The method according to claim 1 wherein the macromolecules are selected from the group consisting of DNA, RNA, cellular metabolites, lipids, carbohydrates and proteins.

3. The method according to claim 1 wherein the macromolecule is DNA and is selected from viral, nucleic, mitochondrial, plastid, bacterial and synthetic.

4. The method according to claim 1 wherein the macromolecule is RNA and is selected from rtRNA, tRNA, miRNA, rRNA and mRNA.

5. The method according to claim 1 wherein the macromolecule is a cellular metabolite and is selected from those produced by a metabolic cycle or enzymatic effects.

6. The method according to claim 1 wherein the macromolecule is lipid and is selected from liposomes, cell membrane lipids, intracellular membrane lipids and extracellular lipids.

7. The method according to claim 1 wherein the macromolecule is carbohydrate and is selected from protein-bound carbohydrates and nucleic acid-bound carbohydrates.

8. The method according to claim 1 wherein the macromolecule is protein and is selected from intracellular and extracellular.

9. The method according to claim 3 wherein the modification is selected from bisulfite and biotinylation.

10. The method according to claim 4 wherein the modification is selected from fluorination and methylation.

11. The method according to claim 6 wherein the modification is selected from heating, liposome formation, micelle formation, uni-layer formation and bilayer formation.

12. The method according to claim 7 wherein the modification is selected from oxidation, de-oxidation, amination and de-amination.

13. The method according to claim 8 wherein the modification is selected from phosphorylation, dephosphorylation, methylation, biotinylation, amination, deamination, glycosylation and deglycosylation.

14. The method according to claim 1 wherein the sample is selected from tissue, body fluid, a biopsy sample, and preserved tissue.

15. The method according to claim 1 wherein the sample is tissue and is selected from whole organs, dissected organs, epithelium, neural, gastrointestinal, muscle, cardiac, mucosal and endothelium.

16. The method according to claim 1 wherein the sample is body fluid and is selected from whole blood, plasma, urine, saliva, vitreous and serum.

17. The method according to claim 1 wherein the sample is a biopsy sample and is selected from fine needle aspirate, tissue section and skin sample.

18. The method according to claim 1 wherein the sample is preserved tissue and is selected from fresh frozen, paraffin embedded and preserved in a preservation reagent.

19. The method according to claim 18 wherein the preservation reagent is selected from formalin, RNAlater® and dimethylsulfoxide.

20. The method according to claim 1 wherein the purification is selected from particle-based, precipitation, centrifugation, electrophoretic and charge switch.

21. The method according to claim 20 wherein the purification is particle-based and is selected from affinity, sizing and magnetic.

22. The method according to claim 21 wherein the sizing particle is selected from silica-based and diatomaceous earth.

23. The method according to claim 20 wherein the electrophoretic separation is by size and/or charge.

24. The method according to claim 20 wherein the electrophoretic separation is by a gel formed of low molecular weight polymers and/or capillary.

25. A method of extracting and modifying DNA comprising the steps of
a. obtaining a sample containing DNA;
b. incubating the sample with an amount of a bisulfite and for a time and under conditions sufficient to convert a sufficient amount of the non-methylated cytosine residues in the DNA to uracil resides;
c. applying the DNA in the sample to a column;
d. washing the bound DNA to remove contaminants;
e. incubating the column-bound DNA with a desulfonation reagent for a time and under conditions sufficient for desulfonation to occur;
f. washing the bound DNA to remove the desulfonation reagent; and
g. eluting the bisulfite modified DNA from the column.

26. The method according to claim 25, wherein the DNA is obtained by a method comprising the steps of:
a. obtaining a cell sample; and
b. lysing the cell sample to obtain a lysate.

27. The method according to claim 26 wherein the lysate is from about 0.01 to 30 μg.

28. The method according to claim 27 wherein the lysate is applied directly to the column in step b of claim 25.

29. The method according to claim 25 wherein the lysate is obtained by incubation with a proteinase and/or high salt concentration and/or detergent, sonication, freeze-thaw treatment or mechanical disruption.

30. The method according to claim 25 wherein the bisulfite is sodium bisulfite or meta bisulfite.

31. The method according to claim 25 wherein the incubation conditions of step b are about 1-16 hours at 50-95° C. with or without thermocycling.

32. The method according to claim 25 wherein the desulfonation is performed changing the pH.

33. The method according to claim 32 wherein the desulfonation is performed under basic conditions.

34. The method according to claim 33 wherein the conditions include sodium hydroxide and an alcohol.

35. The method according to claim 34 wherein the alcohol is isopropanol or ethanol.

36. The method according to claim 35 wherein the desulfonation occurs from about 0-30 minutes at about 0° C. to about 50° C.

37. The method according to claim 36 wherein the desulfonation occurs at about 15 minutes.

38. The method according to claim 36 wherein the desulfonation occurs at room temperature.
